(19) United States
(12) Patent Application Publication
(10) Pub. No.: US 2005/0062018 A1
(43) Pub. Date: Mar. 24, 2005

(54) Nematic liquid crystal compound, and liquid crystal composition having high speed and high temperature comprising the same

(76) Inventors: Byeong-Seob Ban, Kyungki-do (KR); Yong-Kuk Yun, Kyungki-do (KR); Bong-Sung Seo, Kyungki-do (KR); Bong-Hee Kim, Seoul (KR); Dong-Sik Sakong, Kyungki-do (KR); Yong-Bai Kim, Seoul (KR)

Correspondence Address: CANTOR COLBURN, LLP, 55 GRIFFIN ROAD SOUTH, BLOOMFIELD, CT, 06002, US

(21) Appl. No.: 10/493,717

(22) PCT Filed: Apr. 25, 2002
(86) PCT No.: PCT/KR02/00768

(30) Foreign Application Priority Data
Oct. 25, 2001 (KR) 2001/65881
Oct. 25, 2001 (KR) 2001/65882
Oct. 25, 2001 (KR) 2001/65883
Oct. 25, 2001 (KR) 2001/65884
Oct. 25, 2001 (KR) 2001/65885
Oct. 25, 2001 (KR) 2001/65886

(51) Int. Cl. C09K 19/30; C07C 25/13; C09K 19/12

(52) U.S. Cl. 252/299.63; 252/299.66; 570/129; 428/1.1

(57) ABSTRACT

The present invention relates to a nematic liquid crystal compound and a liquid crystal composition exhibiting a fast response time over a wide temperature range comprising the same, and more particularly, to a liquid crystal composition with fast response characteristics, and having a high phase transition temperature of liquid crystal, and having a low driving voltage and a nematic phase in a wide temperature range, comprising a nematic liquid crystal compound having an isothionate group and at least one fluorine atom in an aromatic ring of a terminal group.

### BACKGROUND OF THE INVENTION

(a) Field of the Invention

The present invention relates to a nematic liquid crystal compound and a high speed high temperature nematic liquid crystal composition comprising the same, and more particularly to a nematic liquid crystal compound and a high speed high temperature nematic liquid crystal composition comprising the same that has a high liquid crystal phase transition temperature, high birefringence rate, a high elasticity coefficient, and a broad operating temperature range of the nematic phase, and thus can realize a high response speed and is effective for various devices requiring liquid crystal such as an LCD.

(b) Description of the Related Art

A nematic liquid crystal composition is an important material for a liquid crystal display (LCD) used for electronic computers, electronic notebooks, personal computers, etc. Recently the LCDs have become widely spread as a display for high speed information treatment for word processors, personal computers, etc.

Liquid crystal displays are classified as TN (Twisted Nematic), STN (Super Twisted Nematic), and ferroelectric liquid crystal displays of the passive matrix method; and TFT (Thin Film Transistor), MIM (Metal Insulator Metal), and diode liquid crystal displays of the active matrix method, according to operation type.

For an active matrix method, high voltage holding ratio with a low leak current is important because a liquid crystal display is operated with switching devices such as TFTs or MIMs for each pixel. In addition, a liquid crystal display tends to require a higher response speed due to an increase in display information amount and for embodiment of a moving images. However, because known nematic liquid crystal compositions have low response speeds, they are difficult use to embody moving images.

In order to solve these problems, the following requirements should be satisfied. First, in order to improve response speed, the viscosity of liquid crystal material should decrease to a range of 20 to 25 mm²/s. Second, in order to lower operating voltage, the dielectric anisotropy (Δε) should increase to a range of 10 to 15 (35° C., 1 kHz). Third, the liquid crystal material should have a nematic phase over broad temperature range, and preferable the nematic phase temperature range is −30 to 80° C. Forth, birefringence (Δ n) should be 0.20 (25° C.) or more.

In addition, although the LCD has physical advantages of a low weight and size, brightness, one of various factors evaluated in determining picture quality, is weak compared to a CRT (Cathode Ray Tube). As the LCD-TV market continues to attract attention as the market for monitors and new LCDs develops, requirements for high brightness and high response speed become very important.

In order to achieve high brightness in an LCD, the phase transition temperature of liquid crystal should be maintained higher due to tube current in the back light. In addition, for a high response speed, rotation viscosity of the material should decrease or the refractive index of the liquid crystal should increase.

Most LCD products marketed thus far use nematic liquid crystal in the TN, IPS, VA modes, and the phase transition temperature thereof is 70 to 80° C. and the response speed is 20 to 30 ms. However, since the phase transition temperature and response speed are still unsatisfactory, improvement in response speed and an increase in phase transition temperature are needed in order to realize TV application and moving images.

In addition, it is well known that liquid crystal material having a high birefringence (Δ n) and a high elasticity coefficient is required in order to improve electro-optical characteristics of the TN-LCD, STN-LCD, and TFT-LCD. However, although known liquid crystal compounds can improve electro-optical characteristics of liquid crystal material, there still remains problems in chemical safety of liquid crystal material and in the operating temperature range of liquid crystal displays because if liquid crystal compounds are used, birefringence of the mixed liquid crystal increases but the smectic phase easily appears or the operating nematic phase temperature range is narrow.

### SUMMARY OF THE INVENTION

The present invention is made in consideration of the problems of the prior art, and it is an object of the present invention to provide a novel nematic liquid crystal compound and nematic liquid crystal composition by increasing birefringence (Δ n) and elasticity coefficient (K11, K33) without significantly increasing the operating voltage, and broadening the operating temperature range of the nematic phase to make response speed high.

It is another object of the present invention to provide a nematic liquid crystal composition that has a high phase transition temperature of liquid crystal and that can realize a high response speed.

It is another object of the present invention to provide a liquid crystal display with improved electro-optical properties using the nematic liquid crystal composition as a compositional ingredient.

In order to achieve these objects, the present invention provides a nematic liquid crystal compound represented by the following Chemical Formula 1: 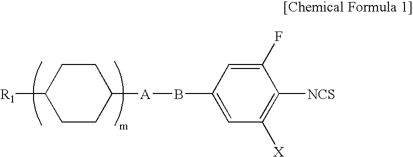
wherein R₁ is CₙH₂ₙ₊₁O, CₙH₂ₙ₊₁, or CₙH₂ₙ₋₁ (n is 1˜15); X is H or F;
   A is 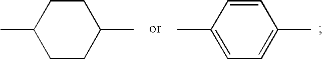
   B is —CH₂—CH₂— or —C≡C—; and m is 0 or 1.

The present invention also provides a high speed high temperature liquid crystal composition comprising the nematic liquid crystal compound of the above Chemical Formula 1.

The present invention also provides a nematic liquid crystal compound represented by the following Chemical Formula 2: 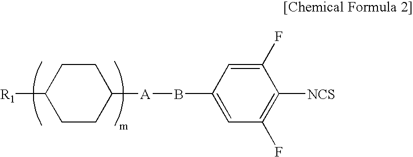
wherein R₁ is CₙH₂ₙ₊₁O, CₙH₂ₙ₊₁ or CₙH₂ₙ₋₁ (n is 1˜15); A is 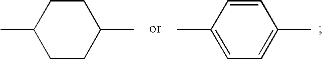
   B is —CH₂—CH₂— or —C≡C—; and m is 0 or 1.

The present invention also provides a nematic liquid crystal composition comprising the nematic liquid crystal compound of the above Chemical Formula 2.

The present invention also provides various liquid crystal displays manufactured using the liquid crystal composition.

### DETAILED DESCRIPTION AND THE PREFERRED EMBODIMENTS

The present invention will now be explained in detail.

The present inventors have developed nematic liquid crystal compounds of the Chemical Formula 1 and 2 and a nematic liquid crystal composition comprising the same, capable of realizing high speed response technology through development of a high temperature high speed liquid crystal, by decreasing viscosity of liquid crystal material, increasing dielectric anisotropy and birefringence, broadening the temperature range of the nematic phase to make response speed higher, and increasing the phase transition temperature of liquid crystal for high brightness and high speed response technology, and completed the present invention.

Particularly, the nematic liquid crystal composition of the present invention can increase the temperature by at least 10° C. compared to the existing commonly used liquid crystal, and realize a response speed of approximately 10 ms, by blending the nematic mixture of the Chemical Formula 1 or 2 as a key material with the existing commonly used liquid crystal composition.

The nematic liquid crystal composition of the present invention comprising the compound of the Chemical Formula 1 or 2 as an essential ingredient has a very high birefringence (Δ n) of 0.20 or more, and a very broad temperature range of the nematic phase of approximately 140° C. or more. Due to these properties, adding a liquid compound substituted with fluorine atoms and substituted with an isothiocyanate group at the end, selected from the group consisting of the compounds of the following Chemical Formula 3 to 6, increases the elasticity coefficient of the nematic liquid crystal composition to accelerate response speed. In addition, since the compound of the Chemical Formula 1 or 2 of the present invention has very good compatibility with the additive compound, a nematic liquid crystal composition showing superior properties can be obtained without loss of good properties of essential ingredients.

The nematic liquid crystal composition of the present invention preferably comprises at least two or more kinds of liquid crystal compounds. The liquid crystal composition of the present invention preferably comprises one or more kinds of compounds selected from the group of the compounds of the Chemical Formula 1 as an essential ingredient. Additionally, the liquid crystal composition of the present invention preferably comprises one or more kinds of compounds selected from the group of the compounds of the Chemical Formula 2 as an essential ingredient.

More preferably, the liquid crystal composition of the present invention further comprises 5 to 40 wt % of one or more kinds of compounds selected from the group consisting of a compound of the following Chemical Formula 3, a compound of the following Chemical Formula 4, a compound of is the following Chemical Formula 5, and a compound of the following Chemical Formula 6, thereby increasing the birefringence (Δ n) and the elasticity coefficient and further accelerating response speed. 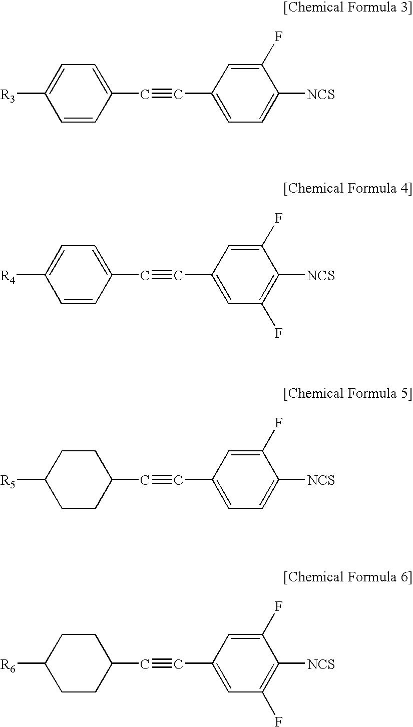
wherein each of R₃˜R₆ is preferably a linear alkyl chain of CₙH₂ₙ₊₁ (n is an integer of 3 to 7) or a linear alkyl chain with one double bond in the middle of an alkyl chain of CH₃CₙH₂ₙ₋₂ (n is an integer of 2 to 6).

Most preferably, the liquid crystal composition of the present invention comprises a commonly used liquid crystal compound together with the liquid crystal compound of the Chemical Formula 1 or 2. Specifically, in order to improve the properties of the liquid crystal composition of the present invention, a generally known nematic liquid crystal, a smectic liquid crystal, a cholesteric liquid crystal, etc. can be mixed with the compound of the Chemical Formula 1 or 2.

In such a case, the compound of the Chemical Formula 1 is preferably one or more kinds selected from the group consisting of compounds of the following Chemical Formula 1a to 1f.

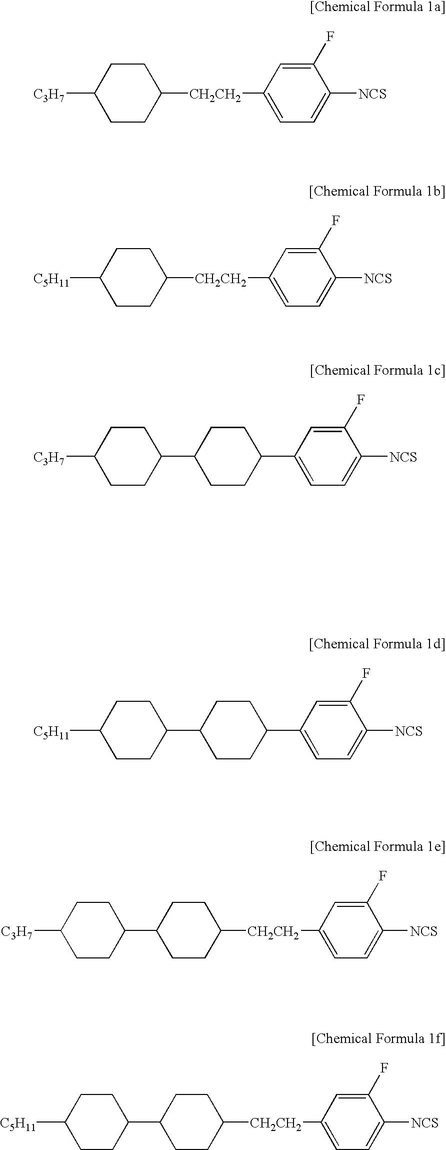

More preferably, the compounds of the Chemical Formula 1 e and 1f are used together. In such a case, the mixing ratio of the compound of the Chemical Formula 1e and the compound of the Chemical Formula 1f is preferably 1 to 80:1 to 80. Still more preferably, the compounds of the Chemical Formulae 1b, 1e, and 1f are used together, and the mixing ratio thereof is preferably 1 to 80:1 to 80:1 to 80.

In addition, the compound of the Chemical Formula 2 is preferably one or more kinds selected from the group consisting of compounds of the following Chemical Formula 2a to 2f:

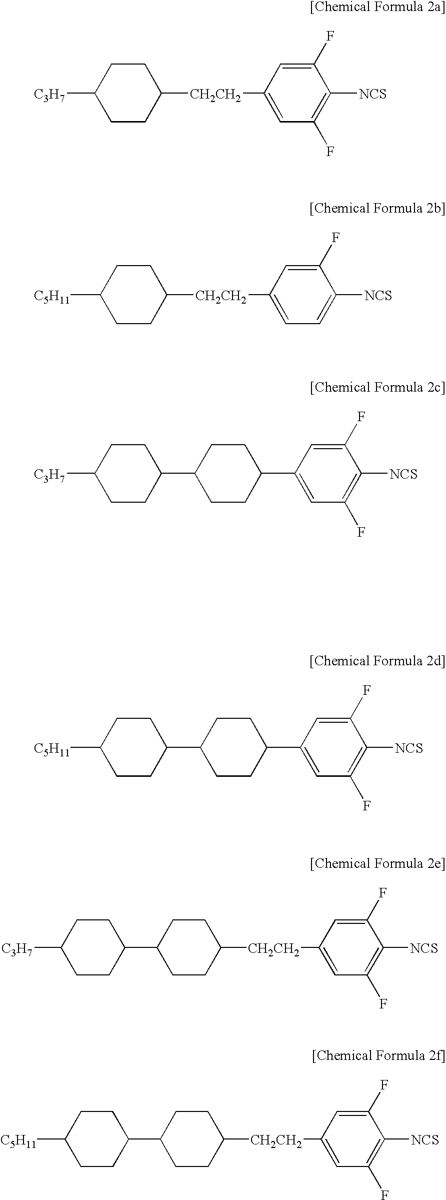

The compound of the Chemical Formula 1 or 2 is preferably contained in an amount of 1 to 90 wt %. If the contents are out of the range, a high phase transition temperature and a high response speed cannot be obtained.

In addition, one or more kinds of the commonly used liquid crystal compounds are preferably used. The commonly used liquid crystal compound is preferably one or more kinds selected from the group consisting of compounds of the following Chemical Formulae 7, 8, and 9. The contents of the commonly used liquid crystal compound are preferably 10 to 99 wt %. 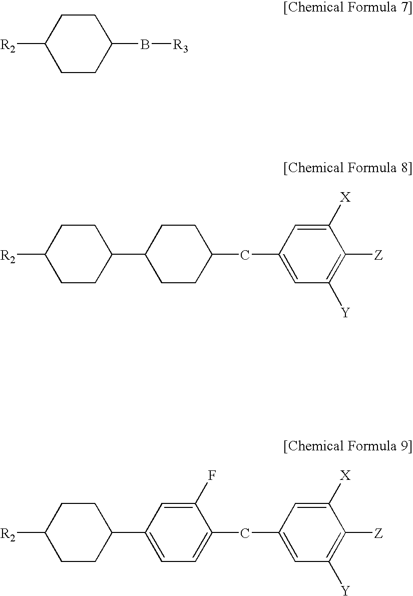
wherein R₂ and R₃ are independently or simultaneously a C₁-C₁₅ alkyl group or alkoxy group; B is a phenyl or a cyclohexyl; C is a single bond, —CH₂CH₂—, or —COO—; X and Y are independently or simultaneously hydrogen or a fluorine atom; and Z is hydrogen, —OCF₃, or a fluorine atom.

As the commonly used liquid crystal compound, the compounds of the above Chemical Formula 7, 8, and 9 can be used in combination. As a preferable example, 4 groups (G1 to G4) of the compounds of the Chemical Formulae 7 to 9 are used together. It is preferable to mix 25 to 45 wt % of G1, 15 to 25 wt % of G2, 10 to 20 wt % G3, and 15 to 30 wt % of G4. The G1 group compounds is at least two kinds selected from the group of the compounds of the Chemical Formula 7; G2 is at least two kinds selected from the group of the compounds of the Chemical Formulae 8 and 9; G3 is at least two kinds selected from the group of the compounds of the Chemical Formula 8; and G4 is at least two kinds selected from the group of the compounds of the Chemical Formulae 8 and 9. However, the mixing ratio is not limited thereto, and it can be modified according to the kinds of the Chemical Formula 1 or Chemical Formula 2.

As mentioned above, since the nematic liquid crystal composition of the present invention comprises one or more kinds of the nematic liquid crystal compounds selected from the group consisting of the compounds of the Chemical Formulae 1 and 2 as essential ingredients, it has high birefringence (Δ n) and a high elasticity coefficient (K11, K33), a low operating voltage, a high response speed, and a large voltage holding ratio.

In addition, the nematic liquid crystal composition of the present invention blends the nematic compound of the Chemical Formula 1 as a key material with the existing commonly used liquid crystal composition, thereby increasing the phase transition temperature by at least 10° C. compared to the existing commonly used liquid crystal, and realizing a low response speed of approximately 10 ms.

Liquid crystal displays can be manufactured by filling the nematic liquid crystal composition of the present invention, if necessary, together with appropriate additives in various display liquid crystal cells. Therefore, various LCD product group devices requiring liquid crystal, preferably a TFT liquid crystal display of the active matrix method, an MIM liquid crystal display of the active matrix method, an IPS (In-plane switching) liquid crystal display of the active matrix method, a simple matrix type twisted nematic liquid crystal display, a simple matrix type super twisted nematic liquid crystal display, a TFT-TN (thin film transistor-twist nematic) display, an AOC (array on color filter), or a COA (color filter on array) liquid crystal display, etc. can be manufactured using the high speed high temperature liquid crystal composition. The manufactured liquid crystal display of the present invention has superior high-speed response and heat resistance, and few after-images in the display screen, and thus can be used under any circumstances.

The present invention will be explained in more detail with reference to the following Examples. However, these are to illustrate the present invention and the present invention is not limited to them.

### EXAMPLE

### Example 1

The phase transition temperatures of the compounds of the Chemical Formula 1 are shown in Table 1. In Table 1, m.p is a temperature at which phase transition from a crystal phase to a liquid crystal phase or isotropic liquid phase occurs, and c.p. is a temperature at which phase transition from a liquid crystal phase to an isotropic liquid phase occurs. For birefringence, the birefringence of 2 ingredients mixed in the liquid crystal at 85 wt % of the mother liquid crystal 4-(4-hexylcyclohexyl)-1-isothiocyanatobenzene and 15 wt % of each compound shown in Table 1 was measured to determine birefringence (Δ n) of the single liquid crystal compound by extrapolation. Each compound was sufficiently purified through distillation, column purification, recrystallization, etc.

TABLE 1

| | | Phase | | |
|---|---|---|---|---|
| | | transition | | |
| | | temperature | | |
| | | (°C.) | | Birefringence |
| No. | Molecular Structural Formula | m.p. | c.p. | (Δn) |
| 1 | 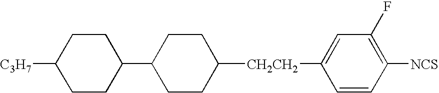 | 72.9 | 189.0 | 0.246 |
| 2 | 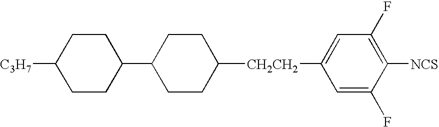 | 81.1 | 175.2 | 0.241 |
| 3 | 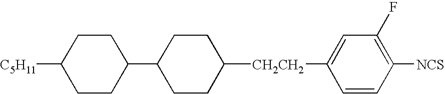 | 60.9 | 186.0 | 0.246 |
| 4 | 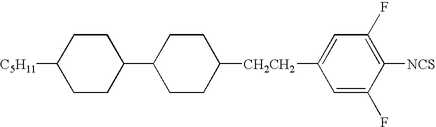 | 50.0 | 175.3 | 0.224 |
| 5 | 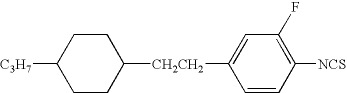 | 50.9 | — | 0.135 |
| 6 | 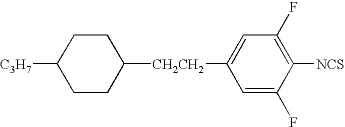 | 33.6 | (20.1) | 0.130 |
| 7 | 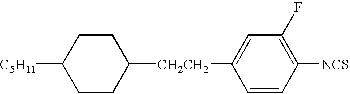 | 29.5 | 44.0 | 0.140 |
| 8 | 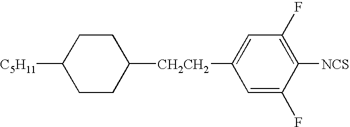 | 9.0 | 33.2 | 0.139 |

### Example 2

A nematic liquid crystal composition was prepared with the following compositional ingredients and ratio. 1 g of a liquid crystal composition was introduced into a test tube and vacuum treated to remove bubbles, and then nitrogen gas was introduced and it was heated at 150° C. for 2 hours to measure the phase transition temperature of the liquid crystal composition. As properties of the liquid crystal composition, T_{NI} (phase transition temperature from nematic phase to isotropic liquid: ° C.), T_{N} (phase transition temperature from solid phase or smectic phase to nematic phase: ° C.), Vth (threshold voltage measured by injecting liquid crystal into a twisted nematic test cell with a distance of 5.7 μm between upper and lower electrodes), [symbol] (ratio of saturation voltage (Vsat) and Vth), and Δ n (birefringence) were measured, and the results are as follows.
Composition of Liquid Crystal 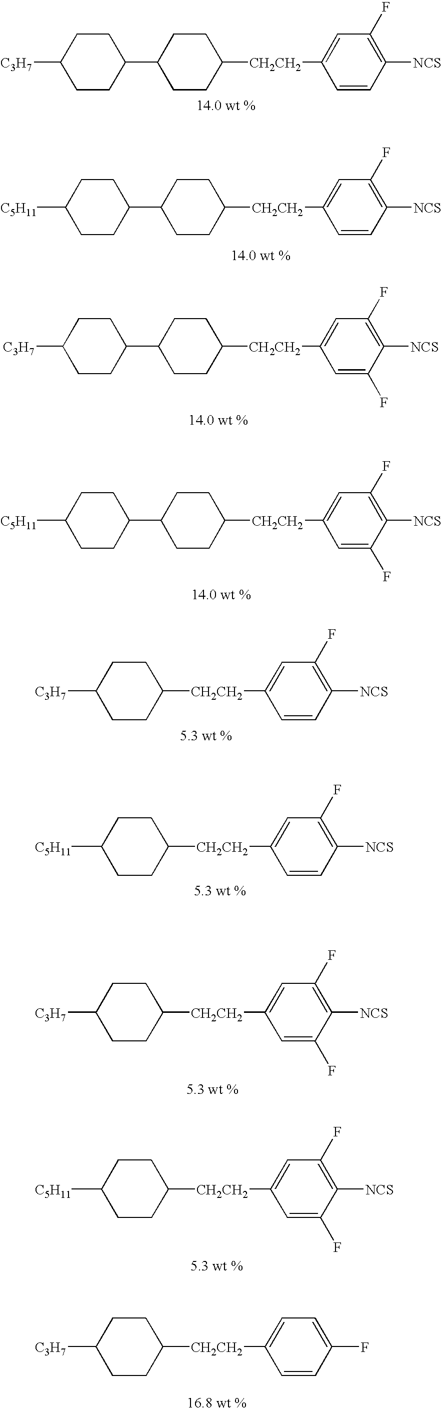
T_{NI} (° C.): 89.6, Δ n: 0.173, Vth (1 kHz, 5V)/V: 1.46, X=1.75, VHR (30 Hz, 25C): 98%

### Comparative Example 1

The commonly used mixture “TM1” with a composition as shown in Table 2 was prepared CTMA=G1+G2+G3+G4). Each contents of G1 to G4 are based on wt %.

TABLE 2

| | | Contents |
|---|---|---|
| | Compound | (wt %) |
| G1 (X is F) | 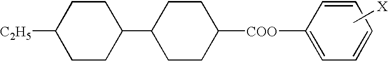 | 3.4 |
| | 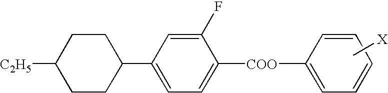 | 10.0 |
| | 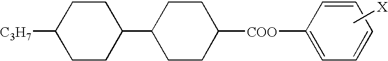 | 10.4 |
| G2 | 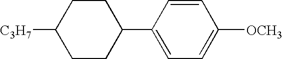 | 6.6 |
| | 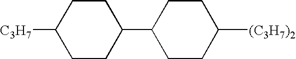 | 5.4 |
| | 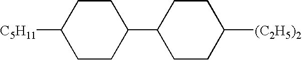 | 20.8 |
| | 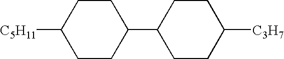 | 5.36 |
| G3 | 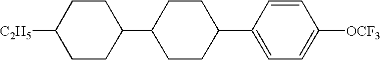 | 7.4 |
| | 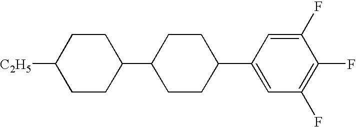 | 7.0 |
| | 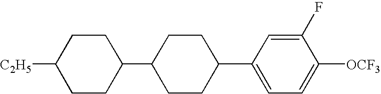 | 2.6 |
| | 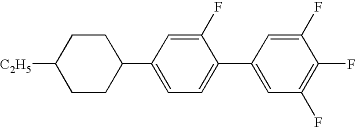 | 3.97 |
| | 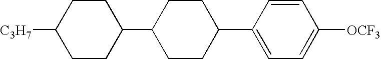 | 2.5 |
| | 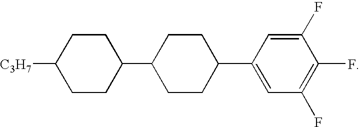 | 3.5 |

The TM1 liquid crystal mixture is presently a commonly used liquid crystal, of which the response speed was measured at 16.2 ms at a cell gap of 4.5 μm, and a phase transition temperature thereof was approximately 80C.

### Examples 3 to 7

In order to confirm changes in the physical properties of high temperature high speed response, a compound of the Chemical Formula 1 c with a high phase transition temperature and a large refractive index anisotropy of the contents as shown in Table 3 as the key material was mixed with the mixed liquid crystal TM1 of Comparative Example 1 as the balance, and the phase transition temperature, the refractive index anisotropy, the dielectric anisotropy, and the response speed (cell gap 3.77 μm) of the liquid crystal were measured according to percentage concentrations, and the results are presented in Table 3.

TABLE 3

| | | | | [Chemical Formula 1c] | |
|---|---|---|---|---|---|
| 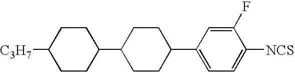 | | | | | |
| | | | | | Response |
| | Contents | T_{NI} | | | Velocity |
| | (wt %) | (° C.) | Δn | Δε | (ms) |
| Comparative | — | 80 | 0.0773 | 5.9 | 16.2 |
| Example 1 | | | | | |
| Example 3 | 7 | 91 | 0.089 | 6.9 | 11 |
| Example 4 | 13 | 100.5 | 0.0100 | 7.7 | 11 |
| Example 5 | 16.9 | 106.8/1086 | 0.107 (20° C.) 0.0958 (28° C.) | 8.2 (20° C.) 6.3 (28° C.) | 11.9 |
| Example 6 | 20 | 111.5 | 0.113 | 8.7 | 12 |
| Example 7 | 26 | 120.9 | 0.123 | 9.5 | 12 |

**denominator: measurement value Example 106.8° C. (calculation value)/106° C. (measurement value)

As can be seen from Table 3, Examples 3 to 7 showed superior results compared to Comparative Example 1 (TM1) without a compound of the Chemical Formula 1c, and particularly, the response speeds decreased to a maximum of 73% and the phase transition temperatures increased to an average of 132%, indicating that the compound is effective for high speed high temperature liquid crystal.

### Example 8

The phase transition temperature, refractive anisotropy, dielectric anisotropy, and response speed (cell gap 3.8 nm) were measured by the same method as in Example 3, except that 18.2 wt % of a compound of the Chemical Formula 1d was used as a key material instead of the compound of the Chemical Formula 1 c. The results are as follows. 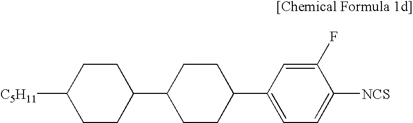
T_{NI}: 106.4° C./106° C., Δ n: 0.1077/0.0937(28° C.), Δ ε: 7.77(25° C.)/6.0(28° C.), response speed:12.3 ms.

As can be seen from the above results, the response speed decreased to 76% compared to TM1 and the phase transition temperature increased to 133%, indicating that the compound is effective for high speed high temperature liquid crystal.

### Comparative Example 2

The commonly used mixture “TM2” with a composition as shown in Table 5 was prepared (TM2=G1+G2+G3+G4). Contents of each of G1 to G4 are based on wt %.

TABLE 5

| | Compound | Contents (wt %) |
|---|---|---|
| G1 | 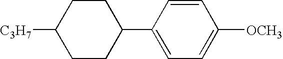 | 6.6 |
| | 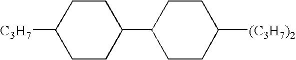 | 5.4 |
| | 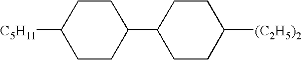 | 20.8 |
| | 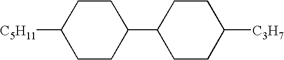 | 5.36 |
| G2 | 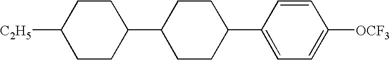 | 7.4 |
| | 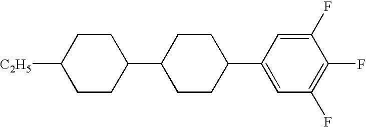 | 7.0 |
| | 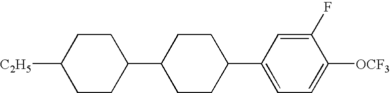 | 2.6 |
| | 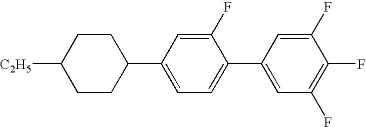 | 3.97 |
| G3 | 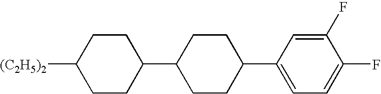 | 11.07 |
| | 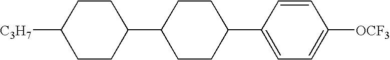 | 3.5 |
| | 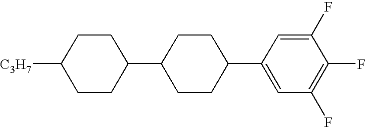 | 2.5 |
| G4 (X is F) | 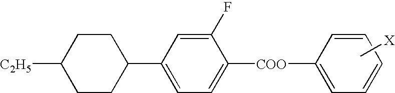 | 3.4 |
| | 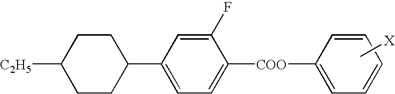 | 10.4 |

The liquid crystal mixture TM2 in Table 5 is presently a commonly used liquid crystal, and a response speed of the liquid crystal was measured as 16.2 ms at a cell gap of 4.6 am, and the phase transition temperature is approximately 80° C.

### Examples 9 to 13

The phase transition temperature, refractive index anisotropy, dielectric anisotropy, and response speed (cell gap 3.75 μm) of liquid crystal were measured according to percentage concentrations by the same method as in Example 3, except that the compound of the following Chemical Formula 1 e of the contents as shown in Table 6 as a key material was mixed with the mixed liquid crystal TM2 of the balance. The results were as shown in Table 6.

TABLE 6

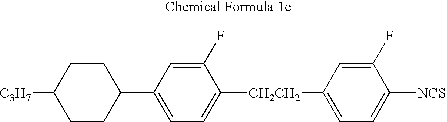

| | Contents | | | Δε | Response |
|---|---|---|---|---|---|
| | (wt %) | T_{NI} (° C.) | Δn | (20° C.) | speed (ms) |
| Comparative | — | 80 | 0.0773 | 5.9 | 16.2 |
| Example 2 | | | | | |
| Example 9 | 7.3 | 88 | 0.0875 | 6.70 | 9.2 |
| Example 10 | 13.2 | 94 | 0.0972 | 7.34 | 9.8 |
| Example 11 | 17.1 | 98/102 | 0.1041 (20° C.)/ 0.09 (28° C.) | 7.76(20° C.)/ 6.01(28° C.) | 10.4 |
| Example 12 | 20.6 | 101 | 0.1102 | 8.15 | 10 |
| Example 13 | 26 | 108 | 0.1195 | 8.74 | 10.6 |

**denominator: measurement value Example — 98° C.(Calculation value)/102° C.(Measurement value)

As can be seen from Table 6, Examples 9 to 13 showed superior results compared to Comparative Example 2 (TM2) without the compound of the Chemical Formula 1e. Specifically, the response speeds decreased to an average of 62%, and the phase transition temperatures increased to an average of 123%, indicating that the compound is effective for high speed high temperature liquid crystal.

### Example 14

The phase transition temperature, refractive index anisotropy, dielectric anisotropy, and response speed (cell gap 3.75 μm) of the liquid crystal were measured by the same method as in Example 3, except that 17 wt % of a compound of the following Chemical Formula 1f was used as a key material instead of a compound of the above Chemical Formula 1 e. 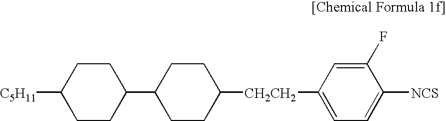
T_{NI}: 98° C./104° C., Δ n:0.1041/0.09(28° C.), Δ ε:/5.9(28° C.), response speed: 11 ms (28° C.)

As can be seen from the results, the response speed decreased to 68% compared to TM2, and the phase transition temperature increased to 130%, indicating the compound is effective for high speed high temperature liquid crystal.

### Comparative Example 3

The commonly used mixture “TM3” was prepared with a composition as shown in Table 7 (TM3=G1+G2+G3+G4). Contents of each of G1 to G4 are based on wt %.

TABLE 7

| | Compound | Contents (wt %) |
|---|---|---|
| G1 (X is F) | 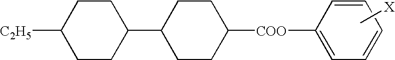 | 3.4 |
| | 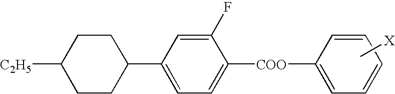 | 10.0 |
| | 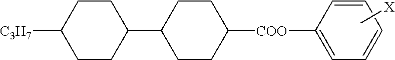 | 10.4 |
| G2 | 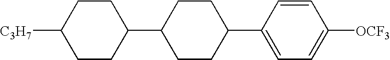 | 2.5 |
| | 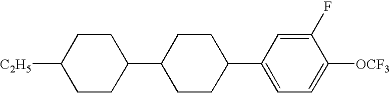 | 2.6 |
| | 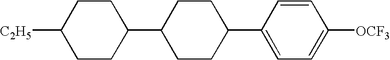 | 7.4 |
| G3 | 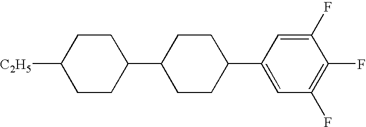 | 7.0 |
| | 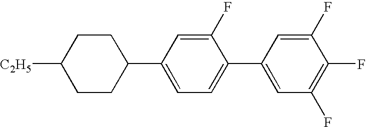 | 3.97 |
| | 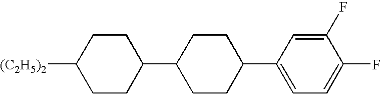 | 11.07 |
| G4 | 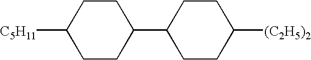 | 20.8 |
| | 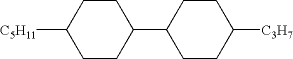 | 5.36 |
| | 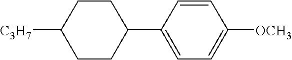 | 6.6 |
| | 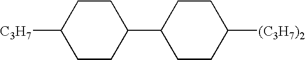 | 5.4 |

The mixed liquid crystal TM3 in Table 7 is presently a commonly used liquid crystal, and the response speed of the liquid crystal was measured to 16˜18 ms at a cell gap of 4.6 μm, and the phase transition temperature was approximately 80° C.

### Examples 15 to 19

The phase transition temperature, refractive index anisotropy, dielectric anisotropy and response speed (cell gap 3.77 μm) of liquid crystal were measured according to percentage concentrations by the same method as in Example 3, except that a compound of the following Chemical Formula 2c of the contents as shown in Table 7 as a key material was mixed with the mixed liquid crystal TM3 of the balance. The results are as shown in Table 8

TABLE 8

| | |
|---|---|
| | [Chemical Formula 2c] |
| 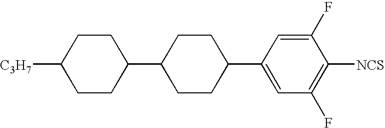 | |

| | Contents | | | | Response |
|---|---|---|---|---|---|
| | (wt %) | T_{NI} (° C.) | Δn | Δε | speed(ms) |
| Comparative | — | 80 | 0.0773 | 5.9 | 16˜18 |
| Example 3 | | | | | |
| Example 15 | 7 | 89 | 0.0866 | 6.9 | 11 |
| Example 16 | 14 | 99 | 0.0984 | 7.9 | 11 |
| Example 17 | 16.7 | 102/101 | 0.1028 (20° C.)/ 0.0958(28° C. | 8.3(20° C.) 6.3(28° C.) | 11.9 |
| Example 18 | 20 | 107 | 0.1084 | 8.7 | 12 |
| Example 19 | 26 | 115 | 0.1184 | 9.6 | 12 |

**denominator: measurement value Example 13 102° C.(Calculation value)/102° C. (Measurement value)

As can be seen from Table 8, Examples 15 to 18 showed superior results compared to Comparative Example 3 (TM3) without a compound of the above Chemical Formula 2c. Specifically, the average response speed decreased to approximately 70% (response speed is expected to be 10 ms or less if the cell gap is controlled to 3.5 μm), and transition temperatures were an average of 22° C. higher than the phase transition temperature of 80° C. of the existing commonly used liquid crystal (127% increase), indicating that the compound is effective for a high speed high temperature liquid crystal with a low cell gap.

### Example 20

The phase transition temperature, refractive index anisotropy, dielectric anisotropy, and response speed (cell gap 3.86 μm) of liquid crystal were measured by the same method as in Example 3, except that 17.8 wt % of a compound of the following Chemical Formula 2d was used as a key material instead of the compound of the Chemical Formula 2c. The results are as follows. 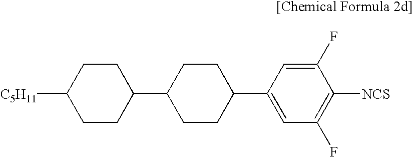
T_{NI}: 101.8° C./101.3° C., Δ n: 0.1027(20° C.)/0.0971(28° C.), Δε: 8.0(28° C.)/6.5(28° C.), response speed: 11.3 ms(28° C.)

If a cell gap is controlled to 3.5 μm, a response speed is expected to be 10 ms or less. As can be seen from the results, the response speed decreased to 66% compared to TM3, and the phase transition temperature increased to 127%, indicating that the compound is effective for high speed high temperature liquid crystal.

### Comparative Example 4

The commonly used mixture “TM4” was prepared with a composition as shown in Table 9 (TM4=G1+G2+G3+G4). Contents of G1 to G4 are based on wt %.

TABLE 9

| | Compound | Contents (wt %) |
|---|---|---|
| G1 | 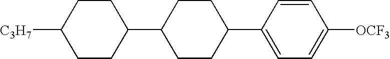 | 2.5 |
| | 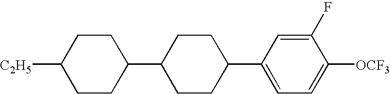 | 2.6 |
| | 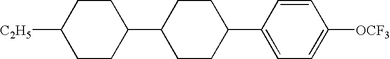 | 7.4 |
| G2 | 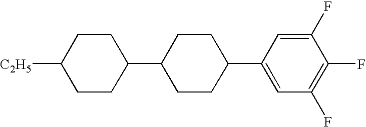 | 7.0 |
| | 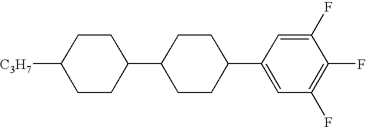 | 3.5 |
| | 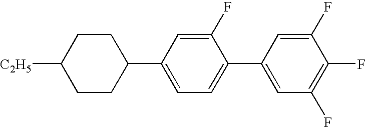 | 3.97 |
| G3 (X is F) | 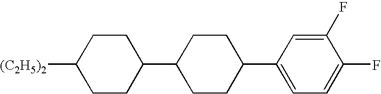 | 11.07 |
| | 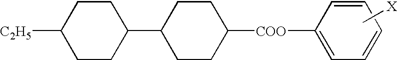 | 3.4 |
| | 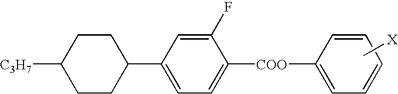 | 10.0 |
| | 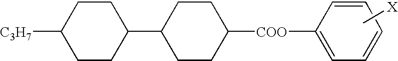 | 10.4 |
| G4 | 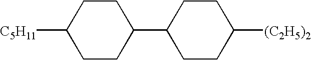 | 20.8 |
| | 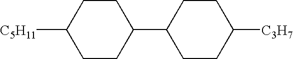 | 5.36 |
| | 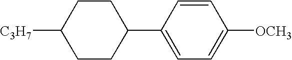 | 6.6 |
| | 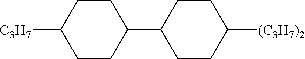 | 5.4 |

The liquid crystal mixture TM4 in Table 9 is presently a commonly used liquid crystal, and the response speed of the liquid crystal was measured to 16.2 ms at a cell gap of 4.6 μm, and the phase transition temperature was approximately 80° C.

### Examples 21 to 25

The phase transition temperature, refractive index anisotropy, dielectric anisotropy, and response speed (cell gap 3.77 μm) of liquid crystal were measured according to percentage concentrations by the same method as in Example 3, except that a compound of the following Chemical Formula 2e of the contents as shown in Table 10 as a key material was mixed with the mixed liquid crystal TM4 of the balance. The results are presented in Table 10.

TABLE 10

| | |
|---|---|
| | [Chemical Formula 2e] |
| 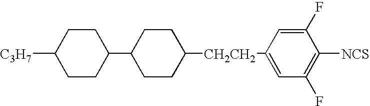 | |

| | | | | | Response |
|---|---|---|---|---|---|
| | Contents | | | Δε | speed |
| | (wt %) | T_{NI} (° C.) | Δn | (20 0C) | (ms) |
| Comparative | — | 80 | 0.0773 | 5.9 | 16.2 |
| Example 4 | | | | | |
| Example 21 | 7 | 87 | 0.0866 | 6.7 | 9.1 |
| Example 22 | 14 | 93 | 0.0982 | 7.5 | 9.7 |
| Example 23 | 16.7 | 96/98.3 | 0.1027(20° C.)/ 0.0907(28° C.) | 7.8(20° C.)/ 6.7(28° C.) | 10.2 |
| Example 24 | 20 | 99 | 0.1082 | 8.2 | 10.3 |
| Example 25 | 26 | 104 | 0.1181 | 8.9 | 10.7 |

**denominator: measurement value Example 96° C.(Calculation value)/98.3° C.(Calculation value)

As can be seen from Table 10, Examples 21 to 25 showed superior results compared to Comparative Example 4 (TM4) without the compound of the Chemical Formula 2e. Specifically, the response speed decreased to 62% and the phase transition temperature increased to 120%, indicating that the compound is effective for high speed high temperature liquid crystal.

### Example 26

The phase transition temperature, refractive index anisotropy, dielectric anisotropy, and response speed (cell gap 3.86 μm) were measured by the same method as in Example 3, except that 17 wt % of a compound of the following Chemical Formula 2f was used as a key material instead of the compound of the above Chemical Formula 2e. The results are as follows.

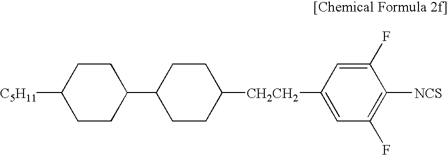

T_{NI}:98° C./100° C., Δn:0.1003/0.0908(28° C.), Δ ε:7.7(20° C.)/6.2(28° C.), response speed: 11 ms (28° C.)

As can be seen from the results, the response speed decreased to 68% compared to TM4 and the phase transition temperature increased to 125%, indicating that the compound is effective for high speed high temperature liquid crystal.

The existing NCS mixture is difficult to use in products because it has a low phase transition temperature and a high refractive index (T_{NI}: 71° C., Δ n: 0.15, response speed: 14.6 ms). In addition, for an NCS mixture with T_{NI}: 95° C., Δ n: 0.089, and response speed: 21.3 ms, it is difficult to apply use for high speed high temperature liquid crystal for moving pictures because it has low response speed. On the other hand, the compounds of Examples of the present invention simultaneously satisfy high speed and high temperature needs and thus it is highly possible to use them in products.

As explained, the nematic liquid crystal compound of the above Chemical Formula 1 or 2 and the liquid crystal composition according to the present invention have high birefringence (Δ n), elasticity coefficient, and dielectric anisotropy (Δ ε); low threshold voltage (Vth) and viscosity; a broad operating temperature range of the nematic phase; a high voltage holding ratio (VHR); and superior chemical stability. Particularly, if used for twisted nematic (TN), super twisted nematic (STN) liquid crystal display (LCD) or active matrix (AM) method thin film transistor (TFT) liquid crystal display devices, etc., after-image and cross talk can be improved, because they have high phase transition temperatures compared to the commonly used liquid crystal and they can realize a high response speed, and they are effective for accelerating response speed and decreasing operating voltage because they have low viscosity and a high elasticity coefficient.

1. A nematic liquid crystal compound represented by the following Chemical Formula 1: 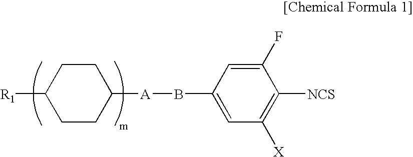
wherein R₁ is CₙH₂ₙ₊₁, CₙH₂ₙ₊₁ or CₙH₂ₙ₋₁, and n is 1˜15; X is H or F; A is 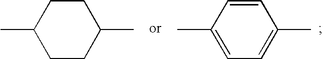
B is —CH₂—CH₂— or —C≡C—; and m is 0 or 1.

2. A nematic liquid crystal composition comprising a nematic liquid crystal compound represented by the following Chemical Formula 1: 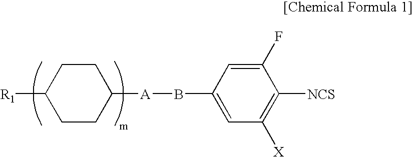
wherein R₁ is CₙH₂ₙ₊₁O, CₙH₂ₙ₊₁ or CₙH₂ₙ₋₁, and n is 1˜15; X is H or F; A is 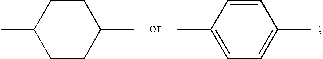
B is —CH₂—H₂— or —C—C—; and m is 0 or 1.

3. The nematic liquid crystal composition according to claim 2, wherein the nematic liquid crystal composition comprises 15 to 40 wt % of one or more kinds of compounds selected from the group consisting of a compound of the following Chemical Formula 3, a compound of the following Chemical Formula 4, a compound of the following Chemical Formula 5, and a compound of the following Chemical Formula 6: 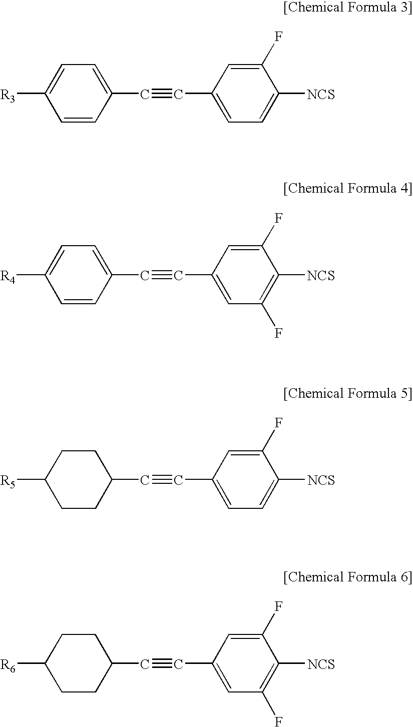
wherein each of R₃˜R₅ is a linear alkyl chain of CₙH₂ₙ₊₁ (n is an integer of 3˜7) or a linear alkyl chain having one double bond in the middle of the alkyl chain CH₃CₙH₂ₙ₋₂ (p is an integer of 2 to 6).

4. The nematic liquid crystal composition according to claim 2, comprising:
a) 1 to 90 wt % of a nematic liquid crystal compound represented by the above Chemical Formula 1; and
b) 10 to 99 wt % of one or more kinds of liquid crystal compounds selected from the group consisting of a compound of the following Chemical Formula 7, a compound of the following Chemical Formula 8, and a compound of the following Chemical Formula 9: 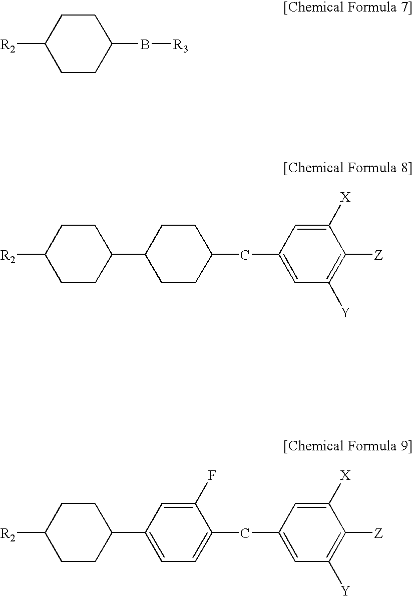
wherein R₂ and R₃ are independently or simultaneously a C₁-C₁₅ alkyl or alkoxy group; B is phenyl or cyclohexyl; C is a single bond, —CH₂CH₂—, or —COO—; X and Y are independently or simultaneously a hydrogen or fluorine atom; and Z is hydrogen, —OCF₃, or a fluorine atom.

5. A nematic liquid crystal compound represented by the following Chemical Formula 2: 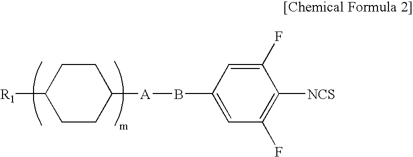
(wherein R₁ is CₙH₂ₙ₊₁O, CₙH₂ₙ₊₁ or CₙH₂ₙ₋₁, and n is 1˜15; A is 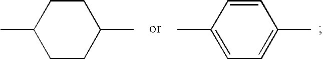
B is —CH₂—CH₂— or —C—C—; and m is 0 or 1.

6. A nematic liquid crystal composition comprising a nematic liquid crystal compound represented by the following Chemical Formula 2: 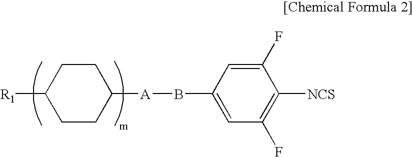
wherein R₁ is CₙH₂ₙ₊₁O, CₙH₂ₙ₊₁ or CₙH₂ₙ₋₁, and n is 1˜15; A is 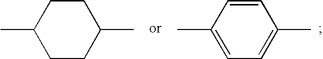
B is —CH₂—CH₂— or —C≡C—; and m is 0 or 1.

7. The nematic liquid crystal composition according to claim 6, wherein the nematic liquid crystal composition comprises 15 to 40 wt % of one or more kinds of compounds selected from the group consisting of a compound of the following Chemical Formula 3, a compound of the following Chemical Formula 4, a compound of the following Chemical Formula 5, and a compound of the following Chemical Formula 6: 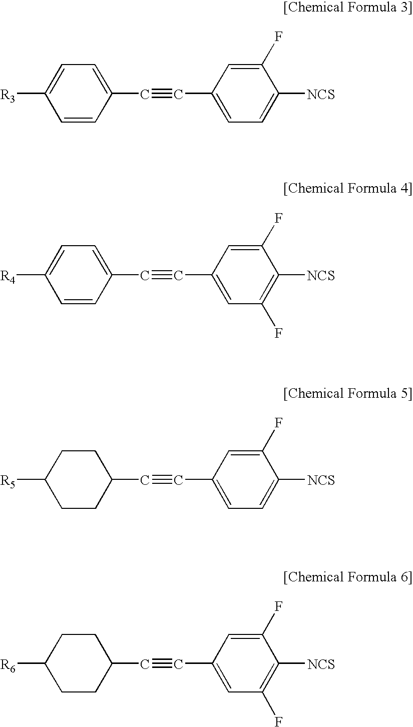
wherein each of R₃R₆ is a linear alkyl chain of CₙH₂ₙ₊₁ (n is an integer of 3˜7) or a linear alkyl chain having one double bond in the middle of the alkyl chain of CH₃CₙH₂ₙ₋₂ (n is an integer of 2 to 6).

8. The nematic liquid crystal composition according to claim 6, comprising:
a) 1 to 90 wt % of the nematic liquid crystal compound represented by the following Chemical Formula 2; and
b) 10 to 99 wt % of one or more kinds of liquid crystal compounds selected from the group consisting of a compound of the following Chemical Formula 7, a compound of the following Chemical Formula 8, and a compound of the following Chemical Formula 9: 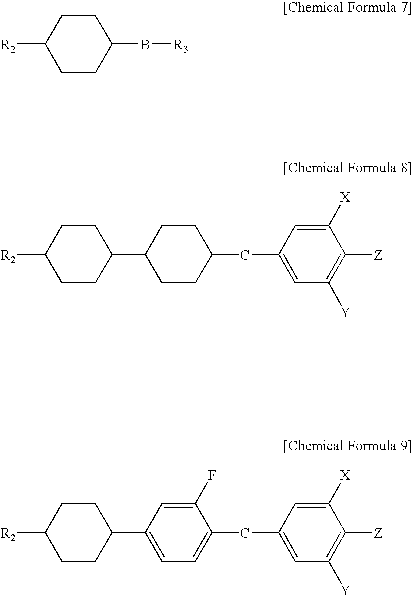
wherein R₂ and R₃ are independently or simultaneously a C₁-C₁₅ alkyl or alkoxy group; B is a phenyl or cyclohexyl; C is a single bond, —CH₂CH₂—, or —COO—; X and Y are independently or simultaneously a hydrogen or fluorine atom; and Z is hydrogen, —OCF₃ or a fluorine atom.

9. A TFT (Thin-film-transistor) liquid crystal display of active matrix method manufactured using the nematic liquid crystal composition of claim 2 or 6.

10. A MIM (Metal-insulator-metal) liquid crystal display of an active matrix method manufactured using the nematic liquid crystal composition of claim 2 or 6.

11. An IPS (In-plane switching) liquid crystal display of an active matrix method manufactured using the nematic liquid crystal composition of claim 2 or 6.

12. A simple matrix type twisted nematic liquid crystal display manufactured using the nematic liquid crystal composition of claim 2 or 6.

13. A simple matrix type super twisted nematic liquid crystal display manufactured using the nematic liquid crystal composition of claim 2 or 6.

14. A TFT-TN (thin film transistor twisted nematic) liquid crystal display manufactured using the nematic liquid crystal composition of claim 2 or 6.

15. An AOC (array on color filter) or COA (color filter on array) liquid crystal display manufactured using the nematic liquid crystal composition of claim 2 or 6.
